(19) United States
(12) Patent Application Publication
(10) Pub. No.: US 2018/0264407 A1
(43) Pub. Date: Sep. 20, 2018

(54) INDUSTRIAL VOC PROCESSING SYSTEM

(71) Applicant: YAO ZHANG, San Leandro, CA (US)

(72) Inventor: YAO ZHANG, SAN LEANDRO, CA (US)

(21) Appl. No.: 15/632,282

(22) Filed: Jun. 23, 2017

Related U.S. Application Data
(63) Continuation-in-part of application No. 15/458,034, filed on Mar. 14, 2017.

(51) Int. Cl. B01D 53/84; F01D 15/10; F01K 25/14; B01D 53/72; C12M 1/00; C12M 1/34

(52) U.S. Cl. CPC B01D 53/84; F01D 15/10; F01K 25/14; B01D 2255/804; C12M 43/04; C12M 41/34; F05D 2220/76; B01D 53/72

(57) ABSTRACT

The present invention provides a system for processing VOC passing through a pipe structure. The pipe structure includes one or more stackable sections for heating decomposition of VOC molecules in the exhaust that passes through the sections and a self-powered cap assembly coupled to the outlet end of the pipe structure. The cap assembly includes a wind turbine mechanically coupled to a generator that supplies electricity to an electronic assembly and to the electrothermal converter in each of the heat decomposition sections. The heat decomposition section includes a paraboloidal heating dish which is coaxially fixed in a cylindric structure with its opening end facing the cap assembly. The electrothermal converter is placed in the focus of the paraboloidal dish. The exhaust gas that passes through the heating decomposition section rotates the turbine that further drives the generator through a shaft. The electricity from the generator activates the electrothermal converter that converts electrical energy into heat energy. The paraboloidal dish reflects the heat from the converter forward into the internal space of cylindric structure. The VOC molecules are decomposed under the high temperature within the cylinder. The system also includes an electronic detecting device and a wireless interface that transmits the VOC data collected by the detecting device to a computer.

### CROSS REFERENCE TO RELATED APPLICATION

This application is a continuation-in-part of and claims the benefit of priority to U.S. patent application Ser. No. 15/458,034, entitled “Industrial VOC Processing System,” filed on Mar. 14, 2017, the content of which is incorporated in its entirety by reference herein.

### FIELD OF THE INVENTION

The present invention generally relates to the technologies of organic waste gas treatment and environmental protection. More particularly, the invention is a system for processing industrial VOC including a first processing section with spraying capacity, a second processing section with biodegradation capacity, a third processing section with thermal decomposition capacity and a self-powered cap assembly.

### BACKGROUND OF THE INVENTION

Volatile organic compounds (VOCs) are emitted as gases from certain solids or liquids. VOCs include a variety of chemicals, some of which may have short and long term adverse health effects. Concentrations of many VOCs are consistently higher indoors (up to ten times higher) than outdoors. VOCs are emitted by a wide array of products numbering in the thousands. Organic chemicals are widely used as ingredients in household products. Paints, varnishes; and wax all contain organic solvents, as do many cleaning, disinfecting, cosmetic, degreasing and hobby products. Fuels are made up of organic chemicals. All of these products can release organic compounds while they are used, and, to some degree, when they are stored. Scientists have discovered that levels of about a dozen common organic pollutants to be 2 to 5 times higher inside homes than outside, regardless of whether the homes were located in rural or highly industrial areas. It is also discovered that while people are using products containing organic chemicals, they can expose themselves and others to very high pollutant levels, and elevated concentrations can persist in the air long after the activity is completed.

The sources of VOC include paints, paint strippers and other solvents, wood preservatives, aerosol sprays, cleansers and disinfectants, moth repellents and air fresheners, stored fuels and automotive products, hobby supplies, dry-cleaned clothing, pesticide, building materials and furnishings, office equipment such as copiers and printers, correction fluids and carbonless copy paper, graphics and craft materials including glues and adhesives, permanent markers and photographic solutions. The sources of industrial sector-based VOC are printing (letterpress, offset and gravure printing processes), wood furniture coating, shoemaking, paint manufacturing and metal surface coating. Among them, benzene and toluene are the major species associated with letterpress printing, while ethyl acetate and isopropyl alcohol are the most abundant compounds of other two printing processes. Acetone and 2-butanone are the major species observed in the shoemaking sector. In the industries of paint manufacturing, wood furniture coating and metal surface coating, aromatics is the most abundant group and oxygenated VOCs is the second largest contributor.

The health effects of VOC may include eye, nose and throat irritation; headaches, loss of coordination and nausea; damage to liver, kidney and central nervous system. Some organics can cause cancer in animals, some are suspected or known to cause cancer in humans. Key signs or symptoms associated with exposure to VOCs include conjunctival irritation, nose and throat discomfort, headache, allergic skin reaction, dyspnea, declines in serum cholinesterase levels, nausea, emesis, epistaxis, fatigue, dizziness. The ability of organic chemicals to cause health effects varies greatly from those that are highly toxic, to those with no known health effect. As with other pollutants, the extent and nature of the health effect will depend on many factors including level of exposure and length of time exposed. Among the immediate symptoms that some people have experienced soon after exposure to some organics include: eye and respiratory tract irritation, headaches, dizziness, visual disorders and memory impairment.

At present, the primary approaches for the treatment of VOCs include catalytic combustion, activated carbon adsorption, low temperature plasma, UV irradiation and so on. The catalytic combustion treatment is relatively more effective, but it requires a high concentration of organic waste gas. Since the concentration of organic gases are usually not high enough for combustion, and natural gas assisted combustion is needed, the operation cost for this approach are relatively high. Activated carbon adsorption method is quite effective. However, it relies on the high cost of activated carbon. Another disadvantage is that the timing for replacement cannot be well controlled, and thus periodical replacement causes waste. The elimination efficiency of organic waste gas by low temperature plasma or by ultraviolet light is quite low.

What is desired is a system, incorporated with an exhaust pipe used in industrial shop or plant, for effectively eliminating VOC in the exhaust by first applying lytic enzyme solution to VOC, then letting certain microbes gnaw the VOC particles, and then decomposing the remaining VOC molecules in the exhaust gas.

### SUMMARY OF THE INVENTION

The present invention provides a system for processing VOC passing through a pipe structure. The pipe structure includes a spraying processing section, a biodegradation processing section, a thermal decomposition processing section, and a self-powered cap assembly coupled to the outlet end of the pipe structure. The thermal decomposition processing section includes one or more stackable cylindric sections for thermal decomposition of VOC molecules in the exhaust that passes through the sections. The cap assembly includes a wind turbine mechanically coupled to a generator that supplies electricity to an electronic assembly and to the electrothermal converter in each of the thermal decomposition sections. The thermal decomposition section includes a paraboloidal heating dish which is coaxially fixed in a cylindric structure with its opening end facing the cap assembly. The electrothermal converter is placed in the focus of the paraboloidal dish. The exhaust gas that passes through the thermal decomposition section rotates the turbine that further drives the generator through a shaft. The electricity from the generator activates the electrothermal converter that converts electrical energy into heat energy. The paraboloidal dish reflects the heat from the converter forward into the internal space of cylindric structure. The VOC molecules are decomposed under the high temperature within the cylinder. The system also includes an electronic detecting device and a wireless interface that transmits the VOC data collected by the detecting device to a computer.

In the first preferred embodiment of this invention, the thermal decomposition processing section includes a cylindric body, a heat collecting plate and a cap assembly coupled to the outlet end. The thermal decomposition processing section can be one or more stackable cylindric sections connected together. Each stackable cylindric section includes a cylindric body and a heating assembly fixed within the cylindric space of the cylindric body. For plugin connection, the caliber of the tube section's one side is smaller than that of the other side. The heating assembly includes a bracket, a reflection cup and an electrothermal converter such as a heating cord. The bracket has three or four legs, at an even angle between the neighboring two legs, with their feet fixed to the interior peripheral wall of the cylindric body. The reflection cup, preferably a paraboloidal dish, is fixed in the center of the bracket. The open end of the reflection cup faces the cap assembly. The electrothermal converter is preferably fixed at the focus of the reflection cup such that the heat generated by the electrothermal converter is reflected to the heating chamber, i.e. the interior space enclosed by the cylindric body. The VOC molecules in the exhaust gas that passes through the chamber are decomposed under the high temperature. The heat collecting plate, fixed at the upper end of the thermal decomposition processing section, collects heat from the decomposition chamber and dissipates the heat downward to the chamber. It can be a flat metal plate with a diameter smaller than the caliber of the cylindric body and has three or four legs, at an even angle between the neighboring two legs, with their feet fixed to the interior peripheral wall of the cylindric body.

In a typical embodiment of the invention, the self-powered cap assembly includes a cylinder and a wind turbine fixed in the cylinder. The wind turbine is coupled to a generator through a shaft. The exhaust gas coming from the thermal decomposition processing section passes through the cylinder and rotates the wind turbine which drives the generator through the shaft. The wind turbine is fixed within the cylinder by a bracket coupled to the interior peripheral wall of the cylinder. The shaft coupled to the wind turbine is coaxial with the cylinder. The generator includes stator consisting of at least a N and a S pole and rotor consisting of a coil assembly coupled to the shaft. In a typical implementation, the magnetic poles are fixed in the interior peripheral walls of the cylinder as a stator. The coil assembly is coupled to the shaft as a rotor. When the exhaust gat passes through, the rotating power from the wind turbine drives the coil assembly to rotate and thus generates electricity. The electric currency from the coil assembly goes through a voltage regulator and then enters an electricity storage device such as a chargeable battery. The storage device supplies electricity to the electrothermal converter that converts the electrical energy to heat energy. The storage device is also electrically coupled to an electronic detecting device placed in the outlet end. The electronic detecting device is electronically coupled to a wireless interface, such as a wireless transmitter, that transmits the VOC data collected by the detecting device to a computer that processes the VOC data.

The detecting device may include an array of electronic sensors fixed on the interior peripheral wall of the cylinder. The sensors collect the VOC data when the exhaust gas passes through outlet end of the cap. If there is no gas that passes through, the sensors are automatically reset to zero. In a typical implementation, the detecting device includes a pendulum rod, two windward leaves, a weight block, and a sensor probe. The pendulum rod is fittingly placed in a longitudinal slot opened on the peripheral wall of the cylinder. The longitudinal slot is along with the axial direction of the cylinder. The measurements of pendulum rod fit the slot's measurements such that the two ends of the pendulum rod can swing around its center. Each windward leaf is vertically coupled to the end of the pendulum rod. The weight block, which can be an iron piece, is fixed to one of the windward leaves. The sensor probe is fixed in the other end of the pendulum rod. If there is no exhaust gas that passes through the cylinder, the pendulum rod is in its first balance status where the windward leaf with the weight block and the sensor prove are outside of the cylinder. However, when exhaust gas passes through the cylinder, the air pressure acts on the windward leaf inside of the cylinder and accordingly the pendulum rod turns such that the sensor probe is inside of the cylinder. Now the pendulum rod is in its second balance status where the windward leaf attached with the weight block is supported by the air pressure from the exhaust gas. The sensor probe, which is above the windward leaf attached with the weight block, collects the data of VOC in the exhaust gas. Due to the shielding of the windward leaf with the weight block, the wind from the exhaust gas does not directly blow on the sensor probe. To avoid unnecessary swing, a resistance piece is attached to each end of the pendulum rod. After a 180 degree turn, the resistance pieces at both ends of the pendulum rod reaches the peripheral wall of the cylinder such that the pendulum rod may not swing inertially. When the wind from the exhaust gas decreases to such a degree that the second balance status cannot sustain due to the gravity of the weight block, the first balance status is restored and accordingly the sensor probe moves to the outside of the cylinder and is reset to zero. The pendulum rod mechanism helps to protect the sensor probe and to reset it after each detection.

In another embodiment of the invention, a generator is coupled to the lower portion of the shaft associated with the wind turbine. The wind from the exhaust gas rotates the turbine which in turn drives the shaft. The generator converts the dynamic energy from the shaft into electrical currency which first goes through a voltage regulator and then enters an electricity storage device. The electricity storage device supplies electricity to the electrothermal converter that provides the heat for thermal decomposition of the VOC molecules in the exhaust gas.

Yet in another embodiment of the invention, the wind turbine is mechanically connected to a transverse transmission device which is mechanically connected to a generator placed outside of the cylinder. The advantage of this implementation is that the generator is not interfered by the high temperature environment within the cylinder.

Yet in another embodiment of the invention, the wind turbine is fixed in the very top end of the cylinder. Through the shaft, the wind turbine drives the generator which is vertically fixed inside of the cylinder. The generator supplies electricity to the electrothermal converter and other electronic devices such as the electronic sensors.

### BRIEF DESCRIPTION OF THE DRAWINGS

FIG. 1 is a schematic diagram illustrating the industrial VOC processing system according to the present invention;

FIG. 2 is a schematic diagram illustrating a multi-unit thermal decomposition processing section incorporated in a cylindric structure;

FIG. 3 is a schematic diagram illustrating a heating assembly that includes a bracket, a reflection cup and an electrothermal converter;

FIG. 4 is a schematic diagram illustrating a heat collecting plate which is fixed at the upper end of the thermal decomposition processing section according to FIG. 2;

FIG. 5 is a schematic diagram illustrating a self-powered cap assembly according to one embodiment of the invention;

FIG. 6 includes FIG. 6A and FIG. 6B, where FIG. 6A is a schematic diagram illustrating the first balance status of the pendulum rod and FIG. 6B is a schematic diagram illustrating the second balance status of the pendulum rod according to the invention;

FIG. 7 is a schematic diagram illustrating the self-powered cap assembly according to second preferred embodiment of the invention;

FIG. 8 is a schematic diagram illustrating the self-powered cap assembly according to a third preferred embodiment of the invention; and

FIG. 9 is a schematic diagram illustrating the self-powered cap assembly according to a fourth preferred embodiment of the invention.

### DETAILED DESCRIPTION OF THE INVENTION

While the present invention may be embodied in many different forms, designs or configurations, for the purpose of promoting an understanding of the principles of the invention, reference will be made to the embodiments illustrated in the drawings and specific language will be used to describe the same. It will nevertheless be understood that no limitation of the scope of the invention is thereby intended. Any alterations and further implementations of the principles of the invention as described herein are contemplated as would normally occur to one skilled in the art to which the invention relates.

The present invention provides an industrial VOC processing system, which includes a first processing section with spraying treatment capacity, a second processing section with biodegradation capacity, a third processing section with thermal decomposition capacity, a self-powered cap assembly with a sensor detection device communicatively coupled to a computer.

The first processing section and the second processing section are incorporated in a pipe structure for exhaust gas in a production shop. The first processing section is coupled to the inlet end of the exhaust pipe and the second processing section is coupled between the first processing section and the third processing section. In the first processing section, the dust and macromolecules in VOC exhaust are eliminated by spraying a cracking solution over the exhaust. In the second processing section, the small molecules in VOC are eliminated by microbes. Under the air pressure of one or more electrical fans, the exhaust gas passes through the first processing section and the second processing section.

As described in the U.S. patent application Ser. No. 15/458,034, the first processing section includes a first section of the pipe where a spraying chamber is installed, a spray device fixed within the upper portion of the spraying chamber, and a cracking tank which is mechanically coupled underneath the first section of the pipe. The cracking tank and the spraying chamber are hydromechanically connected via a first conduit and a second conduit. The cracking tank includes a first pump and a reservoir for containing lytic enzyme solution. The first pump is hydromechanically coupled to the spray device in the first section of the pipe. When the fan is turned on, the exhaust gas is sucked into the chamber through the inlet. At the same time, the first pump pumps the lytic enzyme solution to the spray device via the second conduit. The spray device sprays the lytic enzyme solution over the exhaust that passes through the chamber and the lytic enzyme solution falling to the bottom of the chamber returns to the cracking tank via the first conduit. The lytic enzyme solution is circularly used from the spray chamber to the cracking tank and then to the spray chamber. The exhaust gas that is passing through the spraying chamber is then forced into the second processing section.

The second processing section includes a second section of the pipe constituting a biodegradation chamber and a nutrient supplying tank which is mechanically coupled underneath the biodegradation chamber. The biodegradation chamber and the nutrient supply tank are hydromechanically connected via a third conduit and a fourth conduit. The biodegradation chamber includes an array of pile units for microbial enzymatic hydrolysis. Each pile unit is a rotatable upright post. Microbes that gnaw VOC adhere to the exterior surface of the post. The supplying tank includes a second pump that pumps the nutrient solution up to an upper reservoir in the upper portion of the degradation chamber via the third conduit. The upper reservoir is connected to each pile unit via a microtube or a drip hole. The nutrient solution is supplied to the pile unit periodically. The nutrient solution reaching to the bottom of the degradation chamber returns to the nutrient supplying tank via a fourth conduit. The nutrient solution is circularly used from the supplying tank to the degradation chamber and then to the supplying tank. The VOC in the gas that is passing through the degradation chamber is degraded and eliminated.

Clean air comes out from the outlet of the degradation chamber.

After the treatment in the first and second processing sections, there may be VOC molecules that remain in the exhaust gas. The third processing section is designed to decompose these VOC molecules using thermal decomposition approach.

The third processing section also has a tube structure that can be easily adapted to the outlet of the second processing section. The third processing section has a thermal decomposition capacity using a electrothermal converter which is powered by the electricity generated by the generator that is driven by the wind turbine incorporated in the cap assembly. The cap assembly includes a sensor detection device that is on when exhaust gas passes through the cylindric pass of the cap assembly and that is set zero when no exhaust gas passes.

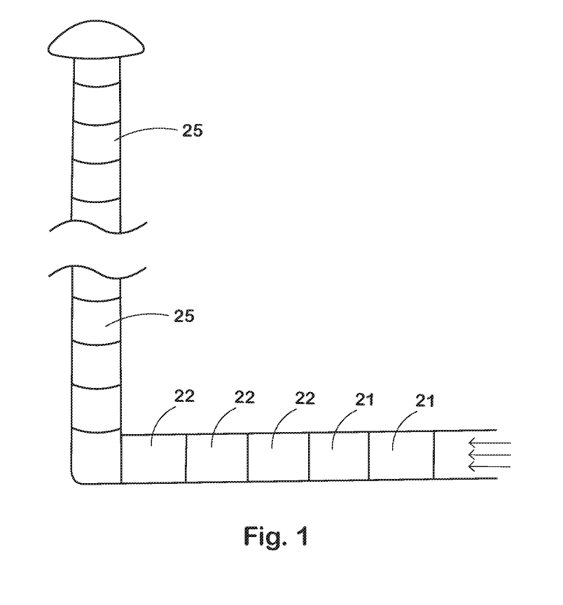

Referring to FIG. 1, which is a schematic diagram illustrating the VOC processing system according to the present invention. The system includes a spraying processing section 21, a biodegradation processing section 22 and a thermal decomposition processing section 25. The thermal decomposition processing section 25 can be one or more stackable cylindric units that can be coupled together. The number of the units can be adjusted according to the VOC processing effects. For easy installation, the stackable units can be coupled via plugin connectors. In certain circumstances, if the thermal decomposition processing section 25 can effectively reduce the VOC, the spraying processing section 21 and the biodegradation processing section 22 may not be needed. In implementation, the section 21 and section 22 are installed in the horizontal portions of the exhaust pipe structure but the thermal decomposition section 25 is preferably installed vertically.

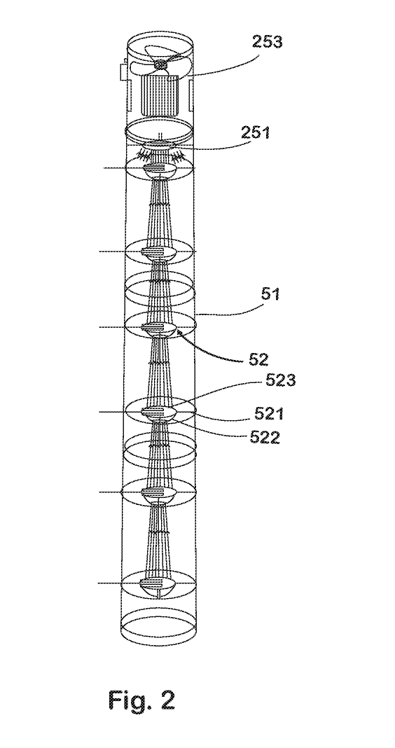

Referring to FIG. 2, which illustrates a multi-unit thermal decomposition processing section 25 incorporated in a vertical cylindric structure. The thermal decomposition processing section 25 includes a cylindric body 51, a heat collecting plate 251, and a self-powered cap assembly 253 coupled to the very last outlet end. The thermal decomposition processing section 25 can be one or more stackable tube sections 252 connected together. Each stackable tube section 252 includes a section of cylindric body 51 and a heating assembly 52 fixed within the cylindric space of the cylindric body 51. For plugin connection, the caliber of the tube section's upper side is smaller than the caliber of the tube section's the lower side.

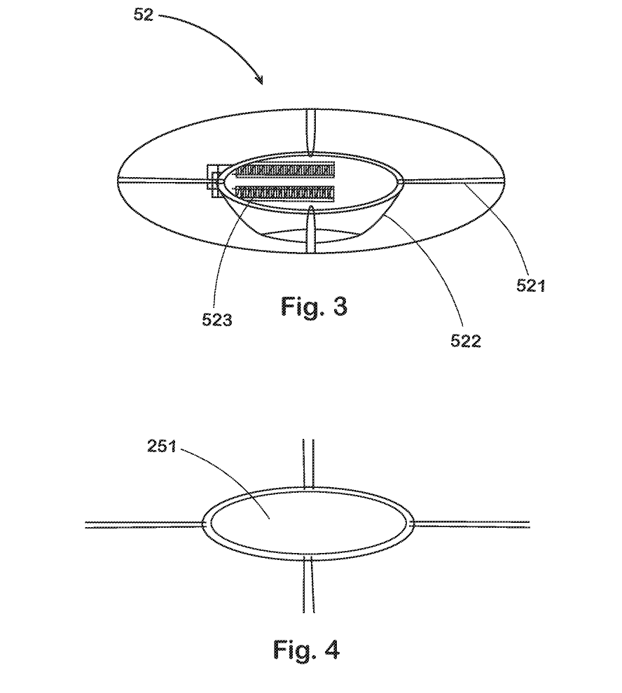

Referring to FIG. 3, which illustrates a heating assembly 52 which is fixed inside of the cylinder. The heating assembly 52 includes a bracket 521, a reflection cup 522 and an electrothermal converter 523 such as a heating cord. The bracket 521 may have three or four legs, at an even angle between the neighboring two legs, with their feet fixed to the interior peripheral wall of the cylindric body 51. The reflection cup 522, preferably a paraboloidal dish, is fixed in the center of the bracket 521. The open end of the reflection cup 522 faces the outlet of the thermal decomposition processing section 25 which is typically installed vertically. The electrothermal converter 523 is preferably fixed at the focus of the reflection cup 522 such that the heat generated by the electrothermal converter is reflected to the heating chamber, i.e. the interior space enclosed by the cylindric body 51. The VOC molecules in the exhaust gas that passes through the chamber are decomposed under the high temperature.

Referring to FIG. 4, which illustrates the heat collecting plate 251 which is fixed at the upper end of the thermal decomposition processing section 25 in FIG. 2. The heat collecting plate 251 collects heat from the decomposition chamber and dissipates the heat downward to the chamber. It also functions to block the heat from dissipating to the gap assembly 253. The heat collecting plate 251 can be a flat metal plate with a diameter smaller than the caliber of the cylindric body 51 and has three or four legs, at an even angle between the neighboring two legs, with their feet fixed to the interior peripheral wall of the cylindric body 51.

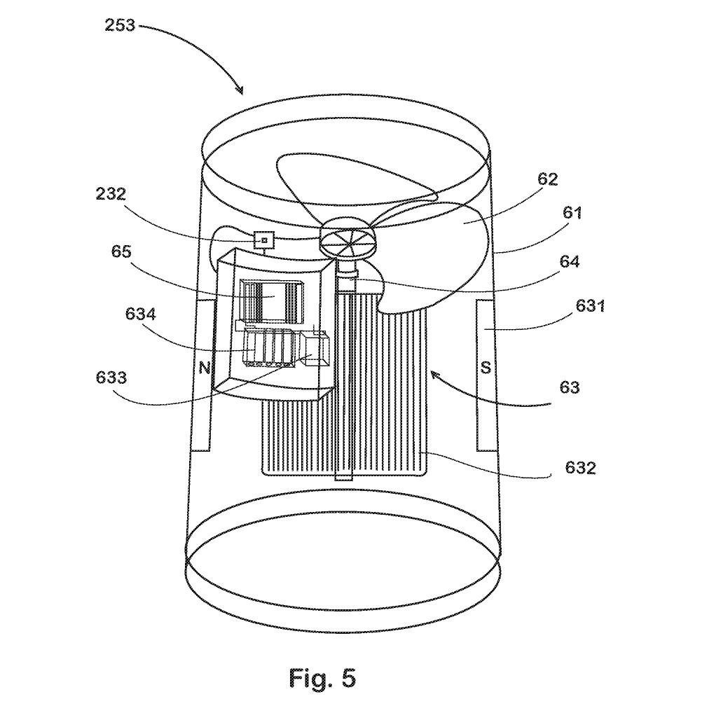

Referring to FIG. 5, which illustrates one preferred embodiment of the self-powered cap assembly 253 according to FIG. 2. The cap assembly 253 includes a cylinder 61 and a wind turbine 62 that is coupled to the generator 63 via the shaft 64. The exhaust gas coming from the thermal decomposition processing section 25 passes through the cylinder 61 and rotates the wind turbine 62 that drives the generator 63 through the shaft 64. The wind turbine 62 is fixed within the cylinder by a bracket coupled to the interior peripheral wall of the cylinder 61. The shaft 64 of the wind turbine 62 is coaxial with the cylinder 61. The generator 63 has at least a stator including a pair of magnetic poles 631 and a rotor including a coil assembly 632 coupled to the shaft 64. In a typical implementation, the magnetic poles 631 are fixed in the interior peripheral walls of the cylinder 61. The coil assembly 632 is coupled to the shaft 64 of the wind turbine 62. When the exhaust gat passes through, the wind turbine 62 rotates the coil assembly 632 and thus generates electricity. The electric currency from the coil assembly 632 goes through a voltage regulator 633 and then enters an electricity storage device 634 such as, for example, a chargeable battery. The electrothermal converter 523 in FIG. 3 is electrically coupled to the storage device 634 such that the electrical currency is converted to heat. The storage device 634 is also electrically coupled to an electronic detecting device 232 placed in the outlet. The electronic detecting device 232 device is electronically coupled to a wireless interface 65, such as a wireless transmitter, that transmits the VOC data collected by the detecting device 232 to a computer server that processes the data.

The detecting device 232 may include an array of electronic sensors. In a typical implementation, the sensors are fixed on the interior peripheral wall of the cylinder 61. The sensors collect the VOC data when the exhaust gas passes through. If there is no gas that passes through, the sensors are automatically reset to zero.

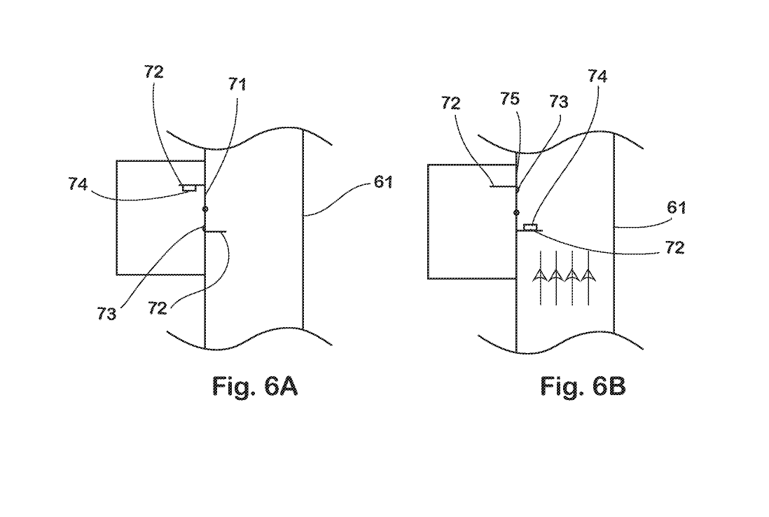

Referring to FIG. 6, which illustrates a mechanical device that includes a pendulum rod 71, two windward leaves 72, a weight block 73 and a sensor probe 74. The pendulum rod 71 is fittingly placed in a longitudinal slot opened on the peripheral wall of the cylinder 61. The longitudinal slot is along with the axial direction of the cylinder 61. The length of pendulum rod 71 is slightly smaller than the slot's length such that the two ends of the pendulum rod 71 can swing freely around its center. Each windward leaf 72 is vertically coupled to an end of the pendulum rod 71. The weight block 73, which can be an iron piece, is fixed to one of the windward leaves 72. The sensor probe 74 is fixed in the other end of the pendulum rod 71. If there is no exhaust gas that passes through the cylinder 61, as shown in FIG. 6A, the pendulum rod 71 is in a first balance status where the windward leaf attached with the weight block 73 resides outside of the cylinder 61 due to the weight, and the sensor probe 74 is also outside of the cylinder 61. However, when the exhaust gas passes through the cylinder 61, the air pressure acts on the windward leaf 72 inside of the cylinder 61, and the pendulum rod 71 turns 180 degrees such that the sensor probe 74 is inside of the cylinder 61 as shown in FIG. 6B. Now the pendulum rod 71 is in a second balance status where the windward leaf attached with the weight block 73 is supported by the air pressure of the exhaust gas that goes upward. The sensor probe 74, which is above the windward leaf attached with the weight block 73, collects the data of VOC in the exhaust gas. Due to the shielding of the windward leaf attached with the weight block 73, the wind from the exhaust gas does not directly blow on the sensor probe 74. To avoid unnecessary swing, a resistance member 75 is attached to each end of the pendulum rod 71. After a 180 degree turn, the resistance member 75 at the two ends of the pendulum rod 71 reaches the peripheral wall of the cylinder 61 such that the pendulum rod 71 may not swing inertially. When the wind from the exhaust gas decreases to such a degree that the second balance status cannot sustain due to the gravity of the weight block 73, the first balance status is restored and thus the sensor probe 74 is outside of the cylinder 61 and is reset to zero. The pendulum rod mechanism helps to protect the sensor probe 74 and to reset it to zero after each detection.

In a typical implementation, a detecting device similar to the detecting device 232 can be installed in the inlet end of the pipe structure such that the data from both the inlet end and outlet end can be compared and analyzed by the computer.

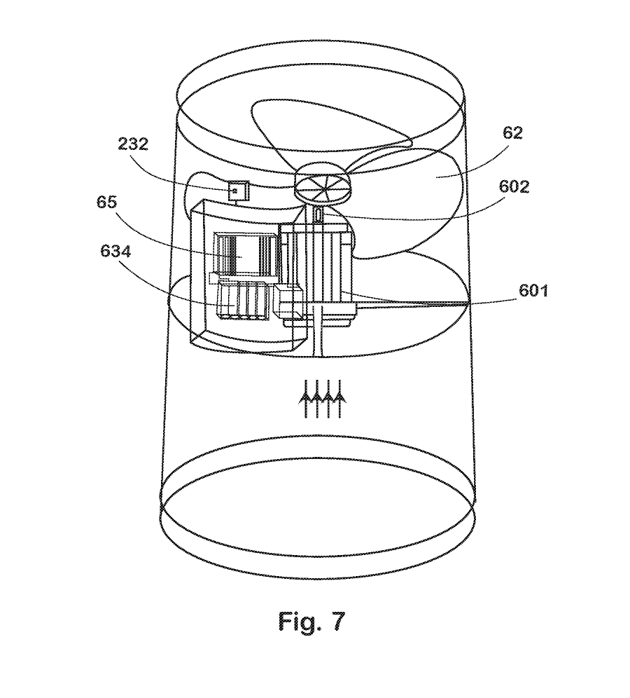

Referring to FIG. 7, which illustrates another preferred embodiment of the cap assembly 253. The N and S magnetic poles fixed in the interior peripheral walls of the cylinder 61 and the coil assembly 632 in FIG. 5 can be replaced by the generator 601 which is coupled at the lower portion of the shaft 64 associated with the wind turbine 62. The wind from the exhaust gas acts on the turbine 62 which rotates the transmission shaft 602. The generator 601 converts the rotating energy from the transmission shaft 602 into electrical currency which first goes through the voltage regulator 633 and then enters the electricity storage device 634. The electricity storage device 634 supplies electricity to the electrothermal converter 523 that provides the heat for thermal decomposition of the VOC molecules in the exhaust gas.

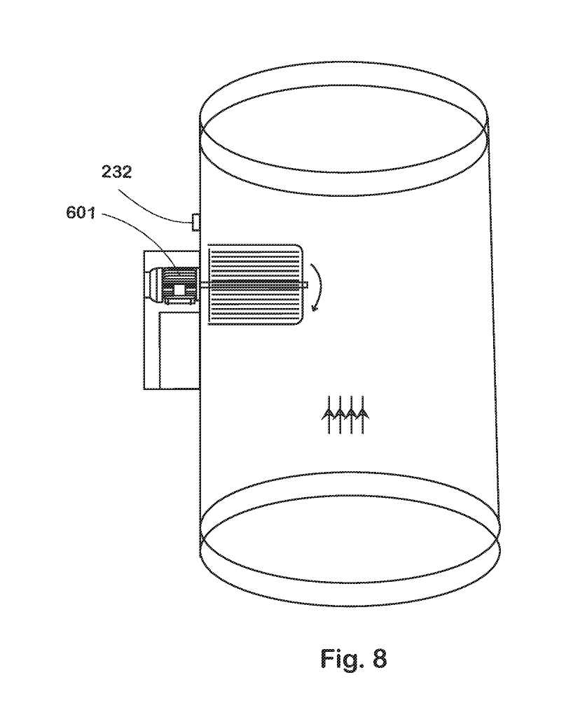

Referring to FIG. 8, which illustrates a third preferred embodiment of the cap assembly 253. The wind turbine (not shown in FIG. 14) is mechanically connected to a transverse transmission device which is mechanically connected to a generator 601 placed outside of the cylinder 61. The advantage of this implementation is that the generator 601 is not interfered by the high temperature environment within the cylinder.

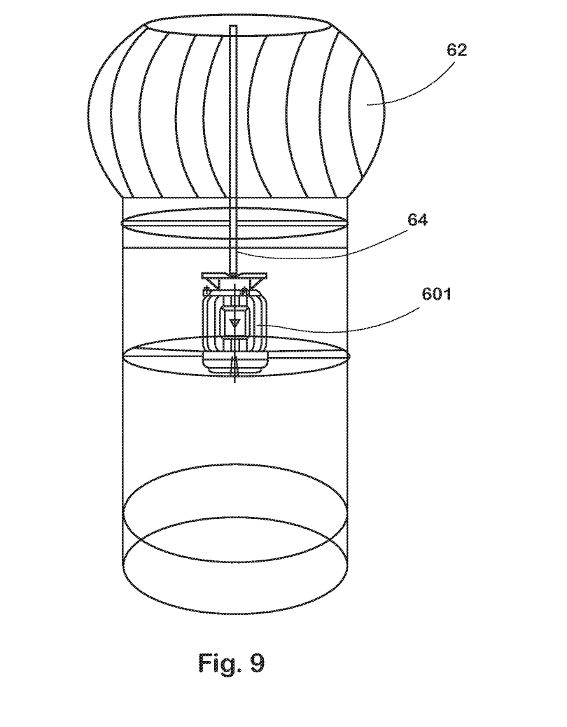

Referring to FIG. 9, which illustrates a fourth preferred embodiment of the cap assembly 253. The wind turbine 62 is fixed in the very top end of the cylinder 61. Through the shaft 64, the wind turbine 62 drives the generator 601 which is vertically fixed inside of the cylinder 61. The generator 601 supplies electricity to the electrothermal converter and other electronic devices such as the electronic sensors.

In summary, the VOC processing system according to the present invention decomposes the VOC molecules in the exhaust gas using high temperature created by the electrothermal converter that is powered by the electricity generated using the wind energy carried by the exhaust gas.

Although one or more embodiments of the newly improved invention have been presented in detail, one of ordinary skill in the art will appreciate the modifications to the coolant in a liquid cooling system for cooling microelectronic components in computer devices with the addition of silver alloy metal. It is acknowledged that obvious modifications will ensue to a person skilled in the art. The claims which follow will set out the full scope of the claims.

1. A system for processing industrial volatile organic compounds (VOC) in industrial exhaust gas, comprising a heat decomposition chamber incorporated in a pipe structure and a cap assembly coupled to said pipe structure's outlet end, wherein said heat decomposition chamber comprises an electrothermal converter fixed in a paraboloidal dish's focus, said paraboloidal dish being coaxially fixed in said pipe structure wherein VOC molecules in the exhaust gas passing through said pipe structure is heat decomposed, wherein said cap assembly comprises a wind turbine coupled to a generator that supplies electricity to said electrothermal converter, and wherein the exhaust gas coming from said heat decomposition chamber rotates said wind turbine.

2. The system of claim 1, further comprising a sensor detection device that is automatically on whenever exhaust gas passes through said cap assembly and that is automatically reset to zero when no exhaust gas passes through said cap assembly.

3. The system of claim 1, wherein said sensor detection device includes a pendulum rod, two windward leaves, a weight block and a sensor probe, wherein said pendulum rod is fittingly placed in a longitudinal slot opened on the peripheral wall of the cylinder of said cap assembly, said longitudinal slot being along with the axial direction of the cylinder, wherein each of said windward leaves is vertically coupled to an end of the pendulum rod, wherein said weight block is fixed to one of said windward leaves, wherein said sensor probe is fixed in the other end of said pendulum rod, wherein when there is no exhaust gas passing through the cylinder, said pendulum rod is in its first balance status where said windward leaf attached with said weight block and said sensor prove reside outside of the cylinder, and wherein when exhaust gas passes through the cylinder, the air pressure acts on said windward leaf inside of the cylinder and accordingly said pendulum rod turns to its second balance status where said windward leaf attached with said weight block is supported by air pressure from said exhaust gas and said sensor probe resides in the cylinder and collects data in said exhaust gas.

4. A system for processing industrial volatile organic compounds (VOC) in industrial exhaust gas, comprising a heat decomposition section incorporated in a pipe structure and a cap assembly coupled to said pipe structure's outlet end, wherein said heat decomposition section comprises two or more heat decomposition chambers stacked together, each of said heat decomposition chambers comprises an electrothermal converter fixed in a paraboloidal dish's focus, said paraboloidal dish being coaxially fixed in a cylindric enclosure of said pipe structure wherein VOC molecules in the exhaust gas passing through said cylindric enclosure is heat decomposed, wherein said cap assembly comprises a wind turbine coupled to a generator that supplies electricity to said electrothermal converter, and wherein the exhaust gas coming from said heat decomposition section rotates said wind turbine.

5. The system of claim 4, further comprising a sensor detection device that is automatically on whenever exhaust gas passes through said cap assembly and that is automatically reset to zero when no exhaust gas passes through said cap assembly.

6. The system of claim 4, wherein said sensor detection device includes a pendulum rod, two windward leaves, a weight block and a sensor probe, wherein said pendulum rod is fittingly placed in a longitudinal slot opened on the peripheral wall of the cylinder of said cap assembly, said longitudinal slot being along with the axial direction of the cylinder, wherein each of said windward leaves is vertically coupled to an end of the pendulum rod, wherein said weight block is fixed to one of said windward leaves, wherein said sensor probe is fixed in the other end of said pendulum rod, wherein when there is no exhaust gas passing through the cylinder, said pendulum rod is in its first balance status where said windward leaf attached with said weight block and said sensor prove reside outside of the cylinder, and wherein when exhaust gas passes through the cylinder, air pressure from the exhaust gas acts on said windward leaf inside of the cylinder and accordingly said pendulum rod turns to its second balance status where said windward leaf attached with said weight block and said sensor probe reside in the cylinder and said windward leaf attached with said weight block is supported by air pressure from said exhaust gas and said sensor collects data in said exhaust gas.

7. A system for processing industrial volatile organic compounds (VOC) in industrial exhaust gas, comprising a pipe structure and a cap assembly coupled to said pipe structure's outlet end, wherein said pipe structure comprises a first processing section, a second processing section and a third processing section, wherein said first processing section comprises a spraying chamber wherein lytic enzyme solution is sprayed over the exhaust gas that passes through said spraying chamber, wherein said second processing section comprises a biodegradation chamber wherein microbial nutrient solution is circularly used for nourishing microbes that gnaw VOC particles in the exhaust gas that enters said biodegradation chamber from said first processing section, wherein said third processing section comprises an electrothermal converter fixed in a paraboloidal dish's focus, said paraboloidal dish being coaxially fixed in a cylindric enclosure wherein VOC molecules in the exhaust gas from said second processing section is heat decomposed, wherein said cap assembly comprises a wind turbine coupled to a generator that supplies electricity to said electrothermal converter in said third processing section, and wherein the exhaust gas coming from said third processing section rotates said wind turbine.

8. The system of claim 7, wherein said cap assembly comprises an array of electronic sensors and a wireless interface that transmits the data collected by said sensors to a computer server, at least one of said sensors being fixed in said pipe structure's inlet end, and at least one of said sensors being fixed in said pipe structure's outlet end.

9. The system of claim 8, wherein said at least one of said sensors being fixed in said pipe structure's outlet end is coupled to one end of a pendulum rod, one end of said pendulum rod being vertically attached to a first windward leaf, the other end of said pendulum rob being vertically attached to a second windward leaf on a direction opposite to said first windward leave, said first windward leave being attached to a weight block, wherein said pendulum rod is fittingly placed in a longitudinal slot opened on the peripheral wall of the cylinder of said cap assembly, said longitudinal slot being along with the axial direction of the cylinder, wherein said at least one sensor is fixed on the same side as said weight block resides but on the opposite end of said pendulum rod, wherein when there is no exhaust gas passing through the cylinder, said pendulum rod is in its first balance status where said first windward leaf attached with said weight block and said sensor prove reside outside of the cylinder, and wherein when exhaust gas passes through the cylinder, air pressure from the exhaust gas acts on said second windward leaf inside of the cylinder and accordingly said pendulum rod turns to its second balance status where said first windward leaf attached with said weight block and said at least one sensor reside in the cylinder and said first windward leaf attached with said weight block is supported by air pressure from said exhaust gas and said at least one sensor collects data in said exhaust gas.

10. The system of claim 9, wherein data collected by said sensors is transmitted to a computer via a wireless interface.
